(19) United States
(12) Patent Application Publication
(10) Pub. No.: US 2022/0241223 A1
(43) Pub. Date: Aug. 4, 2022

(54) USE OF DISULFIRAM OR ITS DERIVATIVES FOR THE TREATMENT OF MITOCHONDRIAL DISEASES OR DYSFUNCTION

(71) Applicants: Universite D'angers, Angers (FR); Centre Hospitalier Universitaire D'angers, Angers (FR); Institut National De La Sante Et De La Recherche Medicale, Paris (FR); Centre National De La Recherche Scientifique, Paris (FR); Universite De Bretagne Occidentale, Brest (FR); Association Francaise Contre Les Myopathies, Paris (FR); Universite De Paris, Paris (FR); Universite Paris-Saclay, Saint-Aubin (FR); Universite De Bordeaux, Bordeaux (FR)

(72) Inventors: Vincent PROCACCIO, Avrille (FR); Agnès RÖTIG, Paris (FR); Agnès DELAHODDE, Verrieres-le-Buisson (FR); Déborah TRIBOUILLARD-TANVIER, Cabanac-et-Villagrains (FR); Geneviève DUJARDIN, Gif-Sur-Yvette (FR); Marc BLONDEL, Brest (FR)

(21) Appl. No.: 17/620,630

(22) PCT Filed: Jun. 19, 2020
(86) PCT No.: PCT/EP2020/067195

§ 371 Date: Dec. 17, 2021

(30) Foreign Application Priority Data
Jun. 19, 2019 (EP) 19305784.1

(51) Int. Cl. A61K 31/145; A61P 3/00

(52) U.S. Cl. CPC A61K 31/145; A61P 3/00

(57) ABSTRACT

The present invention relates to the use of disulfiram or one of its derivatives for use in the treatment of a mitochondrial dysfunction or diseases, advantageously of a genetic mitochondrial disease.

### TECHNICAL FIELD

The present invention provides new pharmacological tools for treating mitochondrial diseases or dysfunction.

### STATE OF THE ART

Mitochondrial diseases are chronic, long-term, mostly genetic, often inherited disorders that occur when mitochondria fail to produce enough energy for the body to function properly. Mitochondrial diseases can be present at birth, but can also occur at any age. It is estimated that 1 in 5000 people has a mitochondrial disease.

Mitochondrial diseases can affect almost any part of the body, including the cells of the brain, nerves, muscles, kidneys, heart, liver, eyes, ears or pancreas. Symptoms of mitochondrial diseases depend on which cells of the body are affected. Patients' symptoms can range from mild to severe, involve one or more organs, and can occur at any age. Symptoms of mitochondrial diseases can include:
Poor growth
Muscle weakness, muscle pain, low muscle tone, exercise intolerance
Vision and/or hearing problems
Learning disabilities, delays in development, mental retardation
Autism, autism-like features
Heart, cardiac dysfunction, cardiac arrhythmia or conduction defects
Liver or kidney diseases
Gastrointestinal disorders, swallowing difficulties, diarrhea or constipation, unexplained vomiting, cramping, reflux
Diabetes
Increased risk of infection
Neurological problems, seizures, migraines, strokes
Movement disorders
Thyroid and/or adrenal dysfunction
Respiratory (breathing) problems
Lactic acidosis (a buildup of lactate)
Dementia.

Mitochondrial dysfunction can also occur when the mitochondria do not work properly, may be due to another disease or condition. Many conditions can lead to secondary mitochondrial dysfunction and affect other diseases, including Alzheimer's or Parkinson's diseases, muscular dystrophy, Lou Gehrig's disease, diabetes and cancer. Individuals with secondary mitochondrial dysfunction do not have primary genetic mitochondrial disease but also suffer from similar symptoms. In addition, some medicines can injure the mitochondria.

The goal of the present treatments is to improve symptoms and slow progression of the disease or dysfunction with e.g. the following recommendations:
Use vitamin therapy
Conserve energy
Pace activities
Maintain an ambient environmental temperature
Avoid exposure to illness
Ensure adequate nutrition and hydration

Moreover and even if most of mitochondrial diseases are of genetic origin, gene therapy seems difficult to implement because of the diversity and complexity of said diseases.

Gill et al. (PLOS ONE, 2018, 13(2)), Mali et al. (Cellular Signalling, 2015, 28(2):1-6), Kuroda et al. (Int. J. Biochem., 1993, 25(1): 87-91) and Hassinen (Biochemical Pharmacology, 1966, 15: 1147-53) have reported in vitro experiments showing that disulfiram tested at very high concentrations (above 25 μM) on normal cells or intact mitochondria affect mitochondrial function. Zhao et al. (CYTOKINE, 2000, 12(9): 1356-67) have reported that disulfiram inhibits TNFα-induced cell death.

Therefore, there is still a need to find new therapeutical approaches for treating said kind of dysfunction or diseases.

### SUMMARY OF THE INVENTION

The inventors have shown that disulfiram (DSF), a pharmacological compound mainly known as an alcohol deterrent because of its action as an inhibitor on aldehyde dehydrogenase, is a potent candidate for treating mitochondrial dysfunction or diseases. The present application reveals that it is efficient at low concentration for a large spectrum of diseases while displaying low toxicity.

### Definitions

The definitions below represent the meaning generally used in the context of the invention and should be taken into account unless another definition is explicitly stated.

In the frame of the invention, the articles “a” and “an” are used to refer to one or several (i.e., at least one) of the grammatical object of the article. By way of example, “an element” means at least one element, i.e. one or more than one elements.

The terms “around”, “about” or “approximately” as used therein when referring to a measurable value such as an amount, a temporal duration and the like should be understood as encompassing variations of ±20% or ±10%, preferably ±5%, more preferably ±1%, and still more preferably ±0.1% from the specified value.

Intervals/ranges: throughout this disclosure, various aspects of the invention can be presented in the form of a value interval (range format). It should be understood that the description of values in the form of an interval is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

“Isolated” means altered or removed from its natural environment or state. For example, an isolated nucleic acid or peptide is a nucleic acid or peptide which has been extracted from the natural environment in which it is usually found whether this be in a plant or living animal for example. A nucleic acid or peptide for example which is naturally present in a living animal is not an isolated nucleic acid or peptide in the sense of the invention whereas the same nucleic acid or peptide partially or completely separated from other components present in its natural environment is itself “isolated” in the sense of the invention. An isolated nucleic acid or protein can exist in substantially purified form, or can exist in a non-native environment such as, for example, a host cell.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics, which are normal or expected for one cell or tissue type, might be abnormal for a different cell or tissue type.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is an animal, preferably a mammal, more preferably a human. It may also be a mouse, a rat, a pig, dog or non-human primate (NHP), such as the macaque monkey.

In the sense of the invention, a “disease” or “pathology” is a state of health of an animal in which its homeostasis is adversely affected and which, if the disease is not treated, continues to deteriorate. Conversely, in the sense of the invention, a “disorder” or “dysfunction” is a state of health in which the animal is able to maintain homeostasis but in which the state of health of the animal is less favourable than it would be in the absence of the disorder. Left untreated, a disorder does not necessarily result in deterioration in the state of health of the animal over time.

A disease or disorder is “alleviated” (“reduced”) or “ameliorated” (“improved”) if the severity of a symptom of the disease or disorder, the frequency with which such a symptom is experienced by the subject, or both of these, is reduced. This also includes the disappearance of progression of the disease, i.e. halting progression of the disease or disorder. A disease or disorder is “cured” (“recovered”) if the severity of a symptom of the disease or disorder, the frequency with which such a symptom is experienced by the patient, or both, is eliminated.

In the context of the invention, a “therapeutic” treatment is a treatment administered to a subject who displays the symptoms (signs) of pathology, with the purpose of reducing or removing these symptoms. As used herein, the “treatment of a disease or disorder” means reducing the frequency or severity of at least one sign or symptom of a disease or disorder experienced by the subject. A treatment is said to be prophylactic when it is administered to prevent the development, spread or worsening of a disease, particularly if the subject does not have or does not yet have the symptoms of the disease and/or for which the disease has not been diagnosed.

As used herein, “treating a disease or disorder” means reducing the frequency or severity of at least one sign or symptom of a disease or disorder experienced by a subject. Disease and disorder are used interchangeably herein in the context of treatment.

In the sense of the invention, an “effective quantity” or an “effective amount” of a compound is that amount of compound which is sufficient to provide a beneficial effect to the subject to which the compound is administered. The expression “therapeutically effective quantity” or “therapeutically effective amount” refers to a quantity which is sufficient or effective to prevent or treat (in other words delay or prevent the development, prevent the progression, inhibit, decrease or reverse) a disease or a disorder, including alleviating symptoms of this disease or disorder.

### DETAILED DESCRIPTION OF THE INVENTION

The present invention relates to the use of disulfiram (DSF) or one of its derivatives, advantageously DSF, for treating mitochondrial dysfunction or a mitochondrial disease.

More specifically and according to a first aspect, the present invention thus relates to a pharmaceutical composition comprising at least disulfiram (DSF) or one of its derivatives, advantageously DSF, for use in the treatment of a mitochondrial disease or mitochondrial dysfunction.

In other words, a composition comprising disulfiram (DSF) or one of its derivatives, advantageously DSF, is used to prepare a medicament intended for the treatment of a mitochondrial disease or mitochondrial dysfunction.

The invention thus relates to a method of treating a mitochondrial disease or mitochondrial dysfunction, comprising administering to a subject in need thereof, at an efficient dose, a composition comprising disulfiram (DSF) or one of its derivatives, advantageously DSF.

Disulfiram (noted DSF), also named tetraethylthiuram disulfide or 1-(diethylthiocarbamoyldisulfanyl)-N,N-diethyl-methanethioamide, is a carbamate derivative. It has the CAS number 97-77-8 and the following formula:

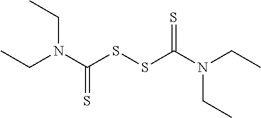 [SMILES: CCN(CC)C(=S)SSC(=S)N(CC)CC]

It is generally in the form of a white powder having high solubility, in e.g. alcohol or chloroform.

It is a member of the dithiocarbamate family comprising a broad class of molecules possessing an R₁R₂NC(S)SR₃ functional group.

Disulfiram, sold under the trade names Antabus® or ESPERAL (tablets containing 500 mg thereof), is a drug used to support the treatment of chronic alcoholism by producing an acute sensitivity to ethanol. Disulfiram works by inhibiting the enzyme acetaldehyde dehydrogenase, causing many of the effects of a hangover to be felt immediately following alcohol consumption. In that context, the usual adult dose is 500 mg orally once a day, generally continued for the first 1 to 2 weeks (initial dose), and then a maintenance dose of 250 mg orally once a day (range: 125 mg to 500 mg once a day). Such a therapy may last months or even years.

Also encompassed by the present invention are derivatives of disulfiram, having the same biological activity, especially as reported in the examples, e.g. on mitochondrial complex I or IV activity or respiration. Of particular interest are the metabolites of DSF.

Examples of such derivatives or metabolites are:
Disulfiram-d20 of formula:

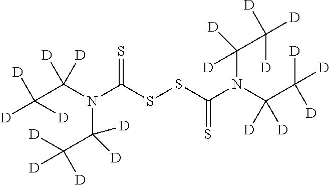 [SMILES: [2H]C([2H])([2H])C([2H])([2H])N(C(=S)SSC(=S)N(C([2H])([2H])C([2H])([2H])[2H])C([2H])([2H])C([2H])([2H])[2H])C([2H])([2H])C([2H])([2H])[2H]]

Diethyldithiocarbamate or DDTC of formula:

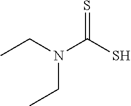

Sodium diethyldithiocarbamate or DEDTC (CAS Number: 148-18-5) of formula:

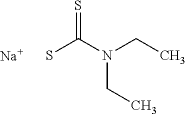

Diethyldithiocarbamate-d10 of formula:

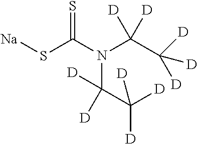

Cu(DEDTC)₂ of formula:

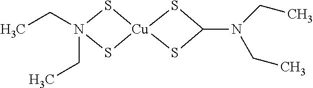

Methyl N,N-diethyldithiocarbamate or DDTC-Me (CAS Number 686-07-07) of formula:

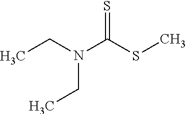

Methyl N,N-diethyldithiocarbamate-d10 of formula:

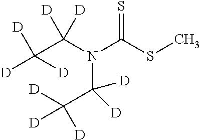 [SMILES: [2H]C([2H])([2H])C([2H])([2H])N(C(=S)SC)C([2H])([2H])C([2H])([2H])[2H]]

Methyl N,N-diethyldithiocarbamoyl sulfoxide or DDTC-MeSO (CAS Number: 145195-14-8), of formula:

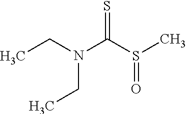

S-methyl N,N-diethylthiocarbamate or DETC-Me (CAS Number: 37174-63-3) of formula:

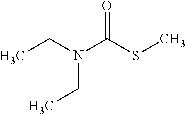

S-methyl N,N-diethylthiocarbamoyl sulfoxide or DETC-MeSO (CAS Number: 140703-15-7) of formula:

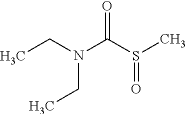

Diethyldithiocarbamate methyl ester sulfine or DDTC-Me Sulfine of formula:

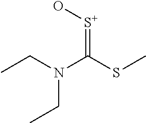

S-methyl N,N-diethylthiocarbamoyl sulfone or DETC-MeSO₂ of formula:

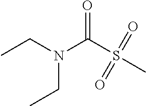

S-methyl N,N-diethyldithiocarbamoyl sulfone or DDTC-MeSO₂
Carbamathione of formula:

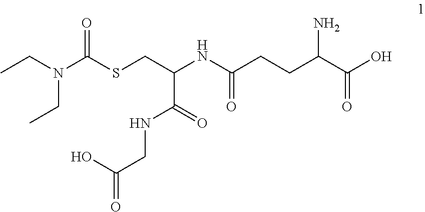 [SMILES: CCN(CC)C(=O)SCC(NC(=O)CCC(N)C(=O)O)C(=O)NCC(=O)O]

Said compounds, including disulfiram, can be further modified to increase their stability, their bioavailability and/or their ability to reach the target tissues, especially mitochondria. As known by the skilled person, said compounds, especially disulfiram, may be present in the composition in a naked form (free) or contained in delivery systems which increase the stability, the targeting and/or the biodisponibility, such as liposomes, or incorporated into carriers such as hydrogels, cyclodextrins, biodegradable nanocapsules, bioadhesive microspheres, vectors or in combination with a cationic peptide.

The present invention also concerns pharmaceutical compositions containing as an active ingredient at least a compound as defined above, as well as the use of this compound or composition as a medicinal product or medicament.

The present invention then provides pharmaceutical compositions comprising a compound according to the invention. Advantageously, such compositions comprise a therapeutically effective amount of said compound, and a pharmaceutically acceptable carrier. In a specific embodiment, the term “pharmaceutically acceptable” means approved by a regulatory agency of the Federal or a state government or listed in the U.S. or European Pharmacopeia or other generally recognized pharmacopeia for use in animals, and humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Saline solutions and aqueous dextrose and glycerol solutions can also be employed as liquid carriers, particularly for injectable solutions. Suitable pharmaceutical excipients include starch, glucose, lactose, sucrose, sodium stearate, glycerol monostearate, talc, sodium chloride, dried skim milk, glycerol, propylene glycol, water, ethanol and the like.

The composition, if desired, can also contain minor amounts of wetting or emulsifying agents, or pH buffering agents. These compositions can take the form of solutions, suspensions, emulsions, sustained-release formulations and the like. Examples of suitable pharmaceutical carriers are described in “Remington's Pharmaceutical Sciences” by E. W. Martin. Such compositions will contain a therapeutically effective amount of the therapeutic, preferably in purified form, together with a suitable amount of carrier so as to provide the form for proper administration to the subject.

In a preferred embodiment, the composition is formulated in accordance with routine procedures as a pharmaceutical composition adapted for e.g. oral administration to human beings. Typically, compositions for oral administration are in the form of tablets, possibly scored tablets or effervescent tablets, further containing excipients suitable for solid dosage form and administration in humans. As an example, available commercial forms of disulfiram are tablets which further contain povidone, magnesium stearate, microcrystalline cellulose, and carmellose sodium. Such tablets can be crushed and mixed with liquids.

Alternatively, the composition may be in a liquid form, advantageously an aqueous composition. Any other suitable solvent can be used.

The amount of the therapeutic agent of the invention, i.e. a compound as disclosed above, which will be effective in the treatment of a disease can be determined by standard clinical techniques. In addition, in vivo and/or in vitro assays may optionally be employed to help predict optimal dosage ranges. The precise dose to be employed in the formulation will also depend on the route of administration, the weight and the seriousness of the disease, and should be decided according to the judgment of the practitioner and each patient's circumstances.

According to a specific embodiment, the composition of the invention is in a solid form, advantageously a tablet, comprising 500 mg of the active compound, in particular disulfiram, or even less. Preferably, the composition comprises a quantity equal to or less than 400 mg, 250 mg, 200 mg or even equal to or less than 100 mg or 50 mg.

According to another embodiment, the composition of the invention is in a liquid form and advantageously comprises less than 1 μM or 500 nM of the active compound, in particular disulfiram, more advantageously between 1 and 100 nM, even more advantageously between 10 and 20 nM.

When used for treating cancer, disulfiram is administered at high (toxic) concentrations so that mitochondria produce free radicals which induce apoptosis and programmed cell death. Advantageously and in the frame of the invention, disulfiram or its derivatives are used at a nontoxic (low) concentration. According to a specific embodiment and as illustrated in the examples below, the toxicity can be evaluated based on lactate production which indicates a switch to glycolysis for energy production instead of mitochondria, advantageously in mutant cybrid cells. High concentrations of lactate are then correlated with drug toxicity of DSF. According to a preferred embodiment, the concentration is less than 1 μM which is considered as toxic for mitochondrial functions, advantageously less than or equal to 900 nM.

Suitable administration should allow the delivery of a therapeutically effective amount of the therapeutic product to the target tissues, depending on the disease.

Available routes of administration are topical (local), enteral (system-wide effect, but delivered through the gastrointestinal (GI) tract), or parenteral (systemic action, but delivered by routes other than the GI tract). In the specific case of mitochondrial diseases, the preferred route of administration of the compositions disclosed herein is generally enteral which includes oral administration. According to other embodiments, it can be a parenteral administration, especially via intramuscular (i.e. into the muscle) or systemic administration (i.e. into the circulating system). In this context, the term “injection” (or “perfusion” or “infusion”) encompasses intravascular, in particular intravenous (IV), and intramuscular (IM) administration. Injections are usually performed using syringes or catheters.

According to one embodiment, the composition is administered orally, intramuscularly, intraperitoneally, subcutaneously, topically, locally, or intravascularly, advantageously orally. According to a preferred embodiment, the composition is for oral administration. Advantageously, the composition is administered orally per os, i.e. by way of the mouth.

As already mentioned, a composition according to the invention is preferably in a solid dosage form adapted for oral administration, advantageously in the form of one or more capsules or tablets. Thus, they can be taken with a little water before or during the main meal.

According to a preferred embodiment, the composition according to the invention is administered daily, for example once per day. The treatment can last several weeks, several months, several years or even for the whole life.

In general, the dosage of therapeutic agent, i.e. disulfiram or one of its derivatives, will vary depending upon such factors as the subject's age, weight, height, gender, general medical condition and previous medical history. Typically, it is desirable to provide the patient with an individual dose of the therapeutic agent which is efficient without being toxic.

According to some embodiments of the invention, the dosage of the composition, advantageously the daily dosage to be taken orally by a human, is inferior or equal to 8 mg/kg or inferior or equal to 7, 6, 5, 4, 3, 2, 1 mg/kg, or even inferior or equal to 0.9, 0.8, 0.7, 0.6, 0.5, 0.4, 0.3, 0.2, or 0.1 mg/kg.

Based on a human weight average of 60 kg for an adult, it means that the dosage of the composition, advantageously the daily dosage to be taken orally, is inferior or equal to 500 mg, or inferior or equal to 450, 400, 350, 300, 250, 200, 150 or 100 mg, or even inferior or equal to 90, 80, 70, 60, 50, 40, 30, 20 or 10 mg.

As already stated, the patient is advantageously a human, particularly a new born, a young child, a child, an adolescent or an adult. The therapeutic tool according to the invention, however, may be adapted and useful for the treatment of other animals, particularly pigs, mice, dogs or macaque monkeys.

As already mentioned, the present invention relates to the treatment of mitochondrial diseases in general, i.e. diseases linked to or caused by mitochondrial dysfunction.

In relation to the examples showing a positive effect of disulfiram or one of its derivatives on the mitochondrial respiratory chain, diseases of particular interest are mitochondrial respiratory chain diseases.

Moreover and as known in the art, most of the mitochondrial diseases are genetic diseases. Genetic diseases are, by definition, diseases resulting from one or a plurality of gene defects (or mutations) in one or a plurality of genes. The gene defects can affect mitochondrial DNA and/or nuclear genes.

The gene defects responsible for the mitochondrial diseases may be point mutations, leading to a codon change. However, the diseases may be linked to the deletion of one or more bases or codons.

Several mitochondrial diseases have been well documented in the prior art:

MELAS syndrome, comprising Mitochondrial myopathy, Encephalopathy, Lactic Acidosis, and Stroke-like episodes, is a genetically heterogeneous mitochondrial disorder with a variable clinical phenotype. The disorder is accompanied by features of central nervous system involvement, including seizures, hemiparesis, hemianopsia, cortical blindness, and episodic vomiting. This syndrome was first associated to the m.3243A>G mutation in mitochondrial DNA, i.e. in the tRNA^{Leu (UUR)} (MTTL1) gene. MELAS syndrome can also be associated with other mitochondrial DNA mutations such as the m.3260A>G mutation. These m.3260A>G or m.3243A>G mutations may also result in other clinical phenotypes including maternally inherited myopathy and cardiomyopathy.

NARP eurogenic Ataxia, Retinitis Pigmentosa) syndrome is caused by mutation in the gene encoding subunit 6 of mitochondrial ATPase (MTATP6 or ATP6). Patients present a variable combination of developmental delay, retinitis pigmentosa, dementia, seizures, ataxia, proximal neurogenic muscle weakness, and sensory neuropathy. This mutation is also associated to Leigh syndrome, a clinically and genetically heterogeneous disorder resulting from defective mitochondrial energy generation. It most commonly presents as a progressive and severe neurodegenerative disorder with onset within the first months or years of life, and may result in early death. Affected individuals usually show global developmental delay or developmental regression, hypotonia, ataxia, dystonia, and ophthalmologic abnormalities, such as nystagmus or optic atrophy. The neurologic features are associated with the classic findings of T2-weighted hyperintensities in the basal ganglia and/or brainstem on brain imaging.

The TAZ gene encodes tafazzin, a mitochondrial transacylase that catalyzes remodeling of immature cardiolipin to its mature composition containing a predominance of tetralinoleoyl moieties. TAZ mutations result in Barth syndrome, an X-linked disease conventionally characterized by dilated cardiomyopathy (CMD) with endocardial fibroelastosis (EFE), a predominantly proximal skeletal myopathy, growth retardation, neutropenia, and organic aciduria, particularly excess of 3-methylglutaconic acid.

The SURF1 gene encodes an assembly factor of mitochondrial complex IV. SURF1 mutations are associated with Leigh syndrome, a progressive and severe neurodegenerative disorder with onset within the first months or years of life, and may result in early death. Affected individuals usually show global developmental delay or developmental regression, hypotonia, ataxia, dystonia, and ophthalmologic abnormalities, such as nystagmus or optic atrophy.

POLG encodes the mitochondrial DNA polymerase, the only polymerase known to be involved in replication of mtDNA. POLG mutations are associated with different clinical presentations transmitted as dominant or recessive traits.

Recessive POLG mutations result in:
Mitochondrial DNA Depletion Syndrome 4A (Alpers Type) characterized by a clinical triad of psychomotor retardation, intractable epilepsy, and liver failure in infants and young children. Pathologic findings include neuronal loss in the cerebral gray matter with reactive astrocytosis and liver cirrhosis. The disorder is progressive and often leads to death from hepatic failure or status epilepticus before age 3 years;
Mitochondrial DNA Depletion Syndrome 4B (MNGIE Type) clinically characterized by chronic gastrointestinal dysmotility and pseudoobstruction, cachexia, progressive external ophthalmoplegia (PEO), axonal sensory ataxic neuropathy, and muscle weakness;
Mitochondrial recessive ataxia syndrome, which includes SANDO (adult onset of sensory ataxic neuropathy, dysarthria, and ophthalmoparesis) and SCAE (spinocerebellar ataxia with epilepsy).

Dominant POLG mutations result in:
Progressive External Ophthalmoplegia (PEO) with Mitochondrial DNA Deletions. The most common clinical features include adult onset of weakness of the external eye muscles and exercise intolerance. Additional symptoms are variable, and may include cataracts, hearing loss, sensory axonal neuropathy, ataxia, depression, hypogonadism, and parkinsonism.

MPV17 encodes a mitochondrial inner membrane protein of unknown function. MPV17 mutations cause:
Mitochondrial DNA depletion syndrome-6, an autosomal recessive disorder characterized by infantile onset of progressive liver failure, often leading to death in the first year of life. Those that survive develop progressive neurologic involvement, including ataxia, hypotonia, dystonia, and psychomotor regression;
Navajo neuropathy: Manifestations include severe anesthesia leading to corneal ulceration, painless fractures, and acral mutilation; muscle weakness; absent or markedly decreased deep tendon reflexes; and normal IQ.

The OPA1 gene encodes a protein that localizes to the inner mitochondrial membrane and regulates several important cellular processes including stability of the mitochondrial network, mitochondrial bioenergetic output, and sequestration of proapoptotic cytochrome c oxidase molecules within the mitochondrial cristae spaces.

Heterozygous OPA1 mutations are associated with dominant optic atrophy with or without mtDNA deletions. Compound heterozygous OPA1 mutations result in
Behr Syndrome that refers to the constellation of early-onset optic atrophy accompanied by neurologic features, including ataxia, pyramidal signs, spasticity, and mental retardation;
Mitochondrial DNA Depletion Syndrome 14 with fatal infantile cardioencephalo-myopathy.

The COA6 gene encodes an assembly factor for mitochondrial cytochrome c oxidase (complex IV). COA6 mutations have been reported in two independent families with fatal infantile cardioencephalomyopathy.

The human BCS1L gene encodes a homolog of S. cerevisiae bcs1 protein involved in the assembly of complex III of the mitochondrial respiratory chain. BCSL1 mutations are associated with:
Mitochondrial Complex III Deficiency, Nuclear Type 1 characterized by neonatal proximal tubulopathy, hepatic involvement, and encephalopathy;
GRACILE Syndrome with growth retardation, amino aciduria, cholestasis, iron overload, lactic acidosis, and early death;
Bjornstad Syndrome characterized by sensorineural hearing loss and pili torti.

The ND6 gene, hosted by the mitochondrial genome, encodes the NADH-ubiquinone oxidoreductase chain 6 protein which is a subunit of the respiratory chain Complex I. The human mutation m.14600G<A, leading to the substitution pPro25Leu, is responsible for mitochondrial diseases (Lin et al.; McManus et al.).

According to a specific embodiment, the diseases to be treated in the frame of the invention are linked to or due to at least one gene defect in at least one of the following genes: MTTL1, ATP6, TAZ, SURF1, POLG, MPV17, OPA1, COA6, ND6 and BCS1L.

Of particular interest is the treatment of a disease selected in the group consisting of: MELAS syndrome, maternally inherited myopathy and cardiomyopathy, NARP syndrome, Leigh syndrome, Barth syndrome, Mitochondrial DNA Depletion Syndrome 4A (Alpers Type), Mitochondrial DNA Depletion Syndrome 4B (MNGIE Type), Mitochondrial recessive ataxia syndrome, Sensory Ataxic Neuropathy Dysarthria and Ophthalmoplegia, Spinocerebellar Ataxia with Epilepsy, Progressive External Ophthalmoplegia, Mitochondrial DNA depletion syndrome-6, Navajo neuropathy, Behr Syndrome, Mitochondrial DNA Depletion Syndrome 14, infantile cardioencephalomyopathy due to cytochrome c oxidase deficiency (COA6 mutations), Mitochondrial Complex III Deficiency Nuclear Type 1, GRACILE Syndrome and Bjornstad Syndrome.

More generally, disulfiram or one of its derivatives can be used to treat mitochondrial dysfunction. Mitochondrial dysfunction, characterized by a loss of efficiency in the electron transport chain and reductions in the synthesis of high-energy molecules such as adenosine-5′-triphosphate (ATP), is a characteristic of aging, and essentially of all chronic diseases.

These diseases include neurodegenerative diseases, such as Alzheimer's disease, Parkinson's disease, Huntington's disease, amyotrophic lateral sclerosis (Lou Gehrig's disease), and Friedreich's ataxia, cardiovascular diseases, such as atherosclerosis and other heart and vascular conditions, diabetes and metabolic syndrome, autoimmune diseases, such as multiple sclerosis, systemic lupus erythematosus, and type 1 diabetes, neurobehavioral and psychiatric diseases, such as autism spectrum disorders, schizophrenia, and bipolar and mood disorders, gastrointestinal disorders, fatiguing illnesses, such as chronic fatigue syndrome and Gulf War illnesses, musculoskeletal diseases, such as fibromyalgia and skeletal muscle hypertrophy/atrophy, muscular dystrophies, cancer, and chronic infections.

According to a specific embodiment, cancer is out of the definition of the diseases to be treated in the frame of the present invention.

According to one aspect, the composition according to the invention is associated with other treatments for the same disease.

According to a specific embodiment, the present invention concerns a composition, advantageously a pharmaceutical composition or a medicinal product containing a compound as described above and potentially other active molecules (other gene therapy proteins, chemical groups, peptides or proteins, etc.) for the treatment of the same disease or a different disease, advantageously of the same disease.

More generally, in relation to mitochondrial diseases, a further compound able to ameliorate mitochondrial function can be administered simultaneously or at different times. In case of simultaneous administration, the two compounds can be associated in the same composition.

Examples of such further compounds are natural supplements, such as L-carnitine, alpha-lipoic acid (α-lipoic acid [1,2-dithiolane-3-pentanoic acid]), coenzyme Q10 (CoQ₁₀ [ubiquinone]), reduced nicotinamide adenine dinucleotide (NADH), membrane phospholipids, possibly in combination.

Examples of compounds used e.g. in the case of MELAS syndrome are Nitric Oxide (NO) precursors such as arginine and citrulline.

According to a specific embodiment, the compound of the invention is not combined with copper or with a salt thereof such as copper gluconate.

Subjects that could benefit from the compositions of the invention include all patients having mitochondrial dysfunction, diagnosed with a mitochondrial disease or at risk of developing such a mitochondrial disease. A subject to be treated can then be selected based on the identification of mutations or deletions in the preferred genes listed above by any method known to the one skilled in the art, including for example gene sequencing, and/or through the evaluation of the corresponding protein expression or activity by any method known to the one skilled in the art.

A target of the invention is to provide a safe (not toxic) treatment. A further aim is to provide an efficient treatment which allows to postpone, slow down or prevent the development of the disease, and possibly to ameliorate the phenotype of the patient which can be easily monitored at the clinical level as disclosed below.

In a subject, the composition according to the invention can be used:
for ameliorating mitochondrial function, especially mitochondrial respiration;
for ameliorating growth;
for ameliorating muscle function;
for ameliorating vision and/or hearing;
for ameliorating respiratory function;
for ameliorating heart, liver or kidney function;
for ameliorating brain function;
for ameliorating digestive function; and/or
for prolonging survival, more generally to ameliorate the quality and the expectancy of life.

According to one aspect, the invention concerns a method for ameliorating mitochondrial function, advantageously without adverse effects, comprising administering to a subject in need thereof a therapeutic quantity of a composition as disclosed above.

Advantageously, said ameliorations are observed for up to 1 month after starting the treatment, or 3 months or 6 months or 9 months, more advantageously for up to 1 year after starting the treatment, 2 years, 5 years, 10 years, or even for the whole life of the subject.

In one embodiment, said ameliorations result in reduced symptom severity and/or frequency and/or delayed appearance.

An amelioration can be evaluated based on methods known in the art, e.g. in the case of MELAS:
assessment of the lactate level, especially cerebral ventricular lactate, as measured e.g. by Magnetic Resonance Spectroscopy (MRS);
assessment of quality and/or expectancy of life by clinical scales, e.g. NMDAS

(Newcastle Mitochondrial Disease Scale for Adults) score or SF-36 (Short Form Health Survey) score;
assessment of the changes in brain e.g. using Magnetic Resonance Imaging (MRI);
assessment of the changes in venous lactate and GDF 15 concentration;
assessment of the changes in mtDNA heteroplasmy in urine and blood.

The adequate parameters for a given case can be adapted depending on the mitochondrial disease.

Thus, the claimed treatment allows improving the clinical state and the various parameters disclosed above in comparison with an untreated subject.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and polypeptides of the invention, and, as such, may be considered in making and practicing the invention. Particularly useful techniques for particular embodiments will be discussed in the sections that follow.

The disclosures of each and every patent, patent application, and publication cited herein are hereby incorporated herein by reference in their entirety.

Without further description, it is believed that one of ordinary skill in the art can, using the preceding description and the following illustrative examples, make and utilize the compounds of the present invention and practice the claimed methods.

### EXAMPLES

The invention and its advantages are understood better from the examples shown below supporting the annexed figures. In particular, the present invention is illustrated with regard to the effect of disulfiram on various yeast models for mitochondrial diseases as well as on some cytoplasmic hybrid (cybrid) cell lines. These examples are not however in any way limiting.

### BRIEF DESCRIPTION OF THE DRAWINGS

FIG. 1:

A/ Effect of DSF on growth of various mutant yeast strains on a respiratory medium as detected by halo tests.

B/ Effect of Sodium diethyldithiocarbamate (DEDTC) and DSF (10 and 30 nmoles) on growth of shy1 mutant yeast strain on a respiratory medium as detected by halo tests.

FIG. 2: Effect of DSF on O₂ consumption rate (VO₂, nmol O₂ 10⁷ cell/min) of various yeast mutant strains, with or without CCCP. The data are the means±SEM of at least three independent experiments. The significance of variations among samples and controls was estimated using Anova multifactorial test: Tukey; (* P value<0.05; ** P<0.01; *** P<0.001; **** P<0.0001)).

FIG. 3: Effect of DSF on NARP cybrid cell lines (JCP239 NARP (T8993G)) grown in glucose deprived medium. DHLA (dihydrolipoic acid) was used as a positive control, as in previous studies (Couplan E et al. & Aiyar R S et al.). Various concentrations of DSF were tested as indicated. After 5 days of treatment by DSF, or DMSO (negative control), or DHLA (positive control), followed by a trypsin treatment, the number of living cells was determined using a Scepter Counter (Millipore). All cell counts were expressed relative to DMSO values set at 100%. This experiment has been repeated two times in triplicates and error bars represent the standard deviation. For each condition, the mean values of the resulting six points is indicated and compared to the negative control (DMSO-treated cells) using the Student's t-test (*P=0.0153; **P=0.0025; ***P<0.0001).

FIG. 4: Determination of the maximal DSF concentration nontoxic for the growth of neuronal MELAS cybrid cells:

A/ Range of DSF concentrations from 90 nM to 900 nM.

B/ Range of DSF concentrations from 5 nM to 40 nM.

FIG. 5: Effect of DSF compared to untreated mutant cells (DMSO vehicle) after 48 h exposure on complex I enzyme activity in MELAS neuronal cybrid cells in low glucose medium (0.5 g/l).

FIG. 6: Effect of DSF on mitochondrial complex I respiration in MELAS neuronal cybrid cells after 48 h exposure in low glucose medium (0.5 g/l).

FIG. 7: Effect of DSF at 10 nM after 48 h exposure on mitochondrial complex I (left) and complex IV (right) respiration in permeabilized MELAS neuronal cybrid cells, in low glucose medium (0.5 g/l).

FIG. 8: Determination of the maximal DSF concentration nontoxic for lactate production in neuronal MELAS cybrid cells. Range of DSF concentrations from 100 nM to 10 μM.

### EXAMPLES 1 TO 3: YEAST MODELS

Each of the various Saccharomyces cerevisiae yeast strains used in examples 1 to 3 contain different specific mutations modeling human mutations resulting in mitochondrial diseases. At different extents, all these yeast strains present growth defect when grown on respiratory medium such as ethanol or glycerol at 28° C. or 36° C. (depending on the strain).

### Yeast Mutant Strains:

A29G→MCC123tRNA^{LeuA30(29)G}: MATα, his3-11, ade2-1, leu2-3,112, ura3-1, trp1-D2, can1-100, syn⁻ (A30(29)G mutation mimics the human m.3260A>G mutation of tRNA^{Leu (UUR)} gene) (De Luca C. et al.)
mip1→DWM-5A: Mat a ade2-1 leu2-3, 112 ura3-1 trp1-1 his3-11, 15 can1-100 Δmip1::KanR transformed by a low copy number plasmid (ARS-CEN) pFL39 (TRP1) expressing the mip1^{G651S} allele synonymous to the human G848S POLG mutation (Baruffini, E. et al.).
bcs1-F401I and shy1-G137R mutants have been constructed in the CW252 strain containing the nuclear background of W303 and an intron-less mitochondrial genome.
coa6 Δ mutant is in the BY4742 background (MATα his3Δ1 leu2Δ0 lys2Δ0 ura3Δ0)
taz1Δ yeast strain was constructed by replacing the open reading frame of TAZ1 by that of TRP1 in the W303-1A strain (MATa ade2-1 ura3-1 his311, 15 trp1-1 leu2-3,112 can1-100) (de Taffin de Tilques et al.).
sym1Δ yeast strain was constructed by replacing the open reading frame of SYM1 by that of kanMX6 in the W303-1A strain (MATa ade2-1 ura3-1 his311, 15 trp1-1 leu2-3,112 can1-100).
mgm1-G430D mutant is in the W303 background (MAT a; ade2-1; leu2-3; his3-11,15; ura3-1; trp1-1; can1-100; mgm1-5_G408(430)D; [Rho+]).
fmc1→MC6 MATa ade2-1 his3-11,15 trp1-1 leu2-3, 112 ura3-1 fmc1::HIS3: [Δi ER OR] for the primary screen and MR14 MATa ade2-1 his3-11,15 trp1-1 leu2-3,112 ura3-1 CAN1 arg8::HIS3 ρ+atp6-L183R or RKY20 MATa ade2-1 his3-11,15 trp1-1 leu2-3,112 ura3-1 CAN1 arg8::HIS3 ρ+atp6-L183P for the secondary screen.

NARP (neuropathy, ataxia, and retinitis pigmentosa) syndrome is caused by various mutations in the mitochondrially-encoded ATP6 gene, which encodes a subunit of ATPase (OXPHOS complex V). The mutations are often heteroplasmic (co-existence of both mutant and wt mitochondrial DNA, mtDNA) within the same cells. Depending both on the type of mutation and on the percentage of mutant mtDNA (degree of heteroplasmy), the clinical outcomes are more or less severe. The ATP6 m.8993T>C/G mutations are among the most frequent in NARP patients and lead to severe forms of the NARP syndrome. A yeast-based assay for the NARP syndrome that identifies drugs potentially active against NARP has been developed by Couplan E et al.. This two-step screening assay is based first, in a primary screen, on the ability of the drug to suppress the respiratory growth defect of the fmc1Δ mutant. FMC1 is a nuclear gene that encodes a protein required at high temperature (35-37° C.) for assembly of the F1 sector of ATP synthase, thereby mimicking the heteroplasmy observed in NARP patients. Indeed, when grown at restrictive temperature (35-37° C.), the mitochondria of the fmc1Δ mutant contain far fewer assembled ATP synthase complexes than a wild-type (WT) strain but the ones that assemble are fully functional. This heterogeneity is also found in patients with decreased levels of ATP synthase due to heteroplasmic ATP6 mutations. Therefore, the fmc1Δ mutant constitutes an appropriate model of these disorders. In a secondary screen, active compounds were tested on various homoplasmic yeast NARP mutants, in particular the equivalent of T8993G and T8993C mutants.

### EXAMPLE 1: EFFECT OF DSF ON GROWTH OF MUTANT YEAST STRAINS GROWN ON NON-FERMENTABLE (RESPIRATORY) MEDIUM

### Materials and Methods

Similarly to an assay previously described (Bach S et al. & Couplan E et al.), the various yeast mutant strains were spread on solid agar-based respiratory (glycerol- or ethanol-based) media and exposed to filters spotted with the tested compound, i.e. Disulfiram noted DSF (Sigma, CAS number: 97-77-8, powder diluted into DMSO). The plates were then incubated at the indicated temperature (which may be 28° C. or 36° C. depending on the strain).

More precisely, 200 μL of the various yeast mutant strain grown in liquid YPD rich fermentable medium (1% Yeast Extract, 0.5% Bacto Peptone, 2% Glucose) at 0.4 OD₆₀₀ were spread on agar-based solid respiratory medium: either YPG (1% Yeast Extract, 0.5% Bacto Peptone, 2% glycerol) or YPE (1% Yeast Extract, 0.5% Bacto Peptone, 2% ethanol). Small sterile filters were then placed on the agar surface and increasing concentrations of DSF were added to the filters at the indicated quantities. The plates were then incubated at 28° C. or 36° C. (depending on the strain) for 4-5 days and then photographed. On the top left, DMSO (the compound vehicle) is used as a negative control.

### Results

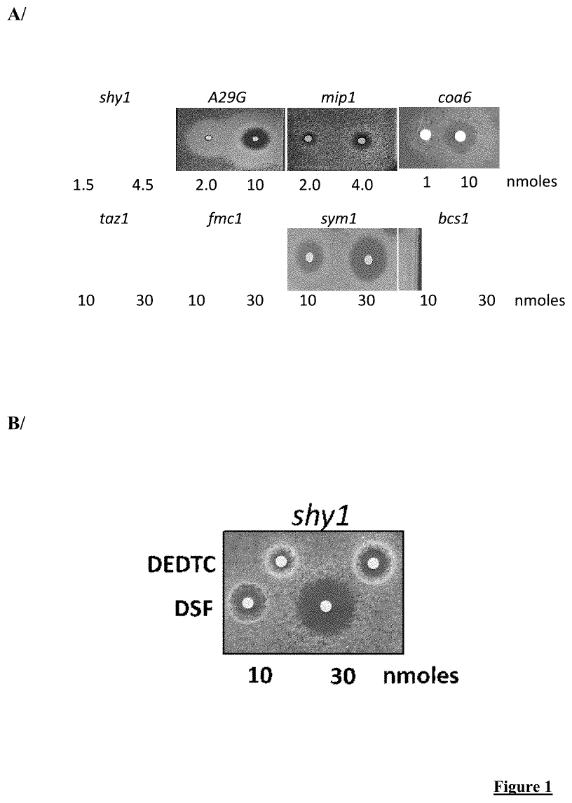

The results are shown in FIG. 1.

The activity of DSF was identified by a halo of enhanced growth around the filter. The advantage of this method is that, in one simple experiment, it allows to test a large range of concentrations due to diffusion of the drug in the growth medium. Hence, this design improves the sensitivity of the screen drastically because active compounds (including DSF, see below) may be toxic at high concentrations. The positive hits obtained were then confirmed using the same experimental procedure.

FIG. 1A reveals that at different extents, DSF suppresses the growth defect on non-fermentable (respiratory) medium of all the tested mutant strains.

FIG. 1B reveals that, at two different concentrations (10 and 30 nmoles), sodium diethyldithiocarbamate (DEDTC), a metabolite of DSF, similarly suppresses the growth defect on non-fermentable (respiratory) medium of shy1 mutant strain.

### EXAMPLE 2: DETERMINATION OF THE MINIMAL DSF CONCENTRATION LEADING TO SUPPRESSION OF THE RESPIRATORY GROWTH DEFECT OF THE VARIOUS MUTANT YEAST STRAINS

### Materials and Methods

Exponentially growing cells were inoculated in fresh non-fermentable YPG or YPE media supplemented, or not, with increasing DSF concentrations. Cell density was determined after 24 or 48 h in order to determine both the optimal concentrations of DSF as for its ability to suppress respiratory growth defect and the concentration at which it displays toxicity.

More precisely, the shy1-G137R and Δcoa6 cells have been grown for 40 h in liquid medium containing 2% glycerol and 0.1% galactose as carbon source and increasing concentrations of DSF (100 nM to 6 μM). The A29G and fmc1Δ cells have been grown for 48 h in liquid medium containing 2% glycerol as carbon source and increasing concentrations of DSF (50 nM to 5 μM). The taz1Δ and sym1Δ cells have been grown for 48 h in rich liquid medium containing 2% ethanol/0.2% galactose and 2% glycerol/2% ethanol as a carbon source, respectively, and increasing concentrations of DSF (100 nM to 9 μM).

Determination of the minimal DSF concentration leading to suppression of the respiratory growth defect of the ATP6, mgm1, mip1 and bcs1 mutant strains has not been investigated.

### Results

The results are shown in Table 1 below.

| | | | | | toxic dose | | optimal | |
|---|---|---|---|---|---|---|---|---|
| yeast gene | | human gene | function | halo | (IC₅₀) | | concentration | |
| ATP6 | mutation | NARP | ATPase | ++ | nd | | nd | |
| FMC1 | deletion | — | ATPase | ++ | 6 | μM | 1 | μM |
| BCS1 | mutation | BCS1L | CIII | +/−− | nd | | nd | |
| SHY1 | Mutation G137R | SURF1 | CIV | ++ | 1.9 +/− 0.1 μM | | 300 | nM |
| COA6 | deletion | COA6 | CIV | +++ | 3.1 +/− 0.1 μM | | 300 nM-1 μM | |
| TAZ1 | deletion | TAZ | CL synthesis | ++ | 9 | μM | 1 | μM |
| MGM1 | mutation TS | OPA1 | mitochondrial | +/− | nd | | nd | |
| | | | dynamics | | | | | |
| SYM1 | deletion | MPV17 | mtDNA | ++ | 9 | μM | 1 | μM |

| | | | maintenance | | | | |
|---|---|---|---|---|---|---|---|
| MIP1 | mutations TS | POLG | mtDNA | +/− | nd | | nd |
| | | | maintenance | | | | |
| A29G | mutation | MELAS | translation | +++ | 400 | nM | 150-200 nM |

nd: not determined

### EXAMPLE 3: EFFECT OF DSF ON YEAST RESPIRATION

### Materials and Methods

The respiratory intensity corresponds to the amount of oxygen consumed relative to time and to the quantity of cells. It reflects the oxidative metabolism of cells. Oxygen consumption was measured using a Hansatech electrode. Cells were grown for 7-8 generations at 28° C. for 24 or 48 h in YPE medium (1% Yeast Extract, 0.5% Bacto Peptone, 2% ethanol) or galactose medium (1% Yeast Extract, 0.5% Bacto Peptone, 2% galactose) supplemented with either DMSO or DSF (200 nM for A29G; 300 nM for shy1 and coati mutants). 10⁷ cells were introduced in the Hansatech electrode at 28° C. 02 consumption was recorded with or without CCCP (Carbonyl Cyanide m-Chloro-Phenyl hydrazine; an uncoupling agent that dissipates the proton gradient that is established during the normal activity of the respiratory chain. In the presence of CCCP, the maintenance of an electrical potential across the inner mitochondrial membrane is impossible and respiration becomes maximal. The 02 consumption rate was calculated based on the linear part of 02 consumption.

### Results

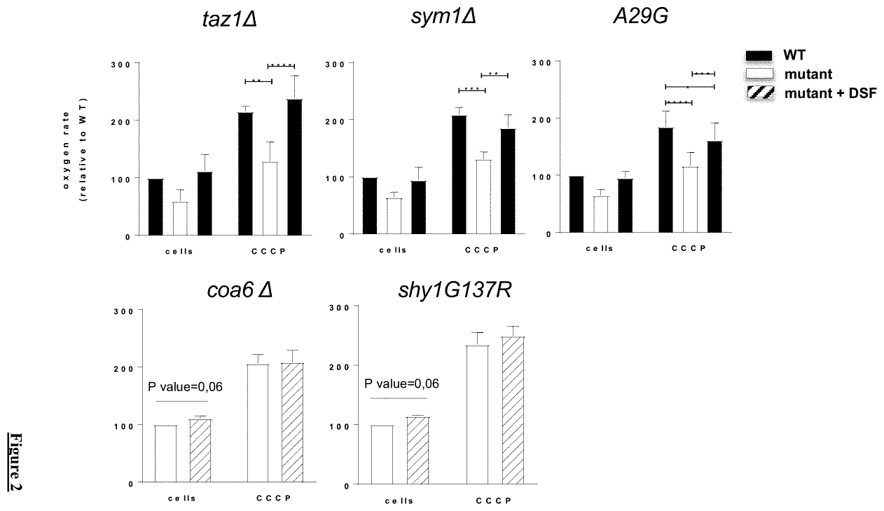

Results are shown in FIG. 2.

It clearly appears that in all cases, DSF increases the yeast respiration. The respiration of the mutant strains treated with DSF can reach the level of respiration observed in the corresponding wild-type strains.

### Examples 4 to 7: Human Cellular Models

As DSF was found to have a positive effect based on various yeast mutant strains, it was then tested on human mutant cells derived from patients: cybrid (cytoplasmic hybrid) cells carrying NARP or MELAS mutations are used in examples 4 to 7.

### EXAMPLE 4: EFFECT OF DSF ON NARP CYBRID RESPIRATORY GROWTH IN LOW GLUCOSE MEDIUM

### Materials and Methods

The cybrid cell lines JCP213 (WT control) and JCP239 (NARP T8993G, Manfredi G et al.) were grown in high glucose (4.5 g/L final concentration) DMEM (supplemented with fetal bovine serum—FBS—at 5% final concentration, sodium pyruvate at 1 mM final concentration, L-glutamine at 4 mM final concentration and uridine at 200 μM final concentration) and then shifted in the same DMEM-based medium except that it is deprived of glucose to encourage the cells to rely on OXPHOS (OXidative PHOSphorylation) rather than glycolysis, supplemented with:
various concentrations of DSF; or
as control, with equivalent quantity of DMSO (compound vehicle, negative control); or
with 150 μM dihydrolipoic acid (DHLA, positive control, as in previous studies (Couplan E et al. & Aiyar R S et al.). All the cells were grown at 37° C. in presence of 5% CO2.

### Results

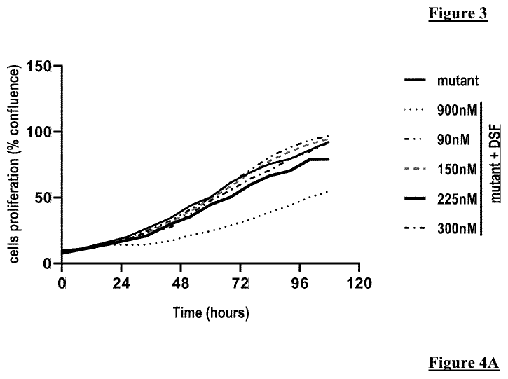

Results are shown in FIG. 3.

DSF, at low concentrations (from 1 nM), has a significant positive effect on the growth of NARP cybrids in glucose-deprived medium. In contrast, at the same range of concentration, DSF has no effect on the growth of control cybrids (JCP213) in glucose-deprived medium.

### EXAMPLE 5: EFFECT OF DSF ON NEURONAL MELAS CYBRIDS

### Materials and Methods

The SH-SY5Y neuronal mutant cybrid cells, carrying the m.3243A>G with 98.6% mutant load responsible for MELAS syndrome, were cultured in standard DMEM high glucose media (4.5 g/L) or in low glucose (0.5 g/L), supplemented with 10% fetal bovine serum, 1% glutamine and 50 μg/ml uridine at 37° C. in presence of 5% CO2 as described elsewhere (Desquiret-Dumas et al. & Geffroy et al.). To optimize drug concentrations, cells were shifted to low glucose-medium 0.5 g/l (to force the cells to rely on OXPHOS rather than glycolysis) supplemented with various concentrations of DSF or of the vehicle (DMSO).

Experiments were done at least in triplicates and error bars represent the standard deviation. Differences between treated cells vs untreated cells (DMSO) were evaluated using the Student's t-test with significant p values<0.05.

### Results

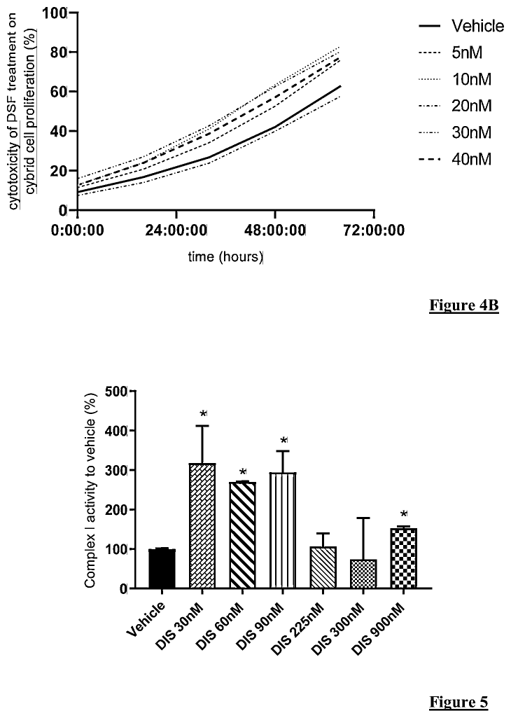

Results are shown from FIGS. 4 to 7.

FIG. 4 reveals that DSF, at concentrations lower than 300 nM, has no impact on cellular growth proliferation of MELAS cybrids, contrary to the 900 nM concentration (FIG. 4A).

### EXAMPLE 6: EFFECT OF DSF ON MITOCHONDRIAL COMPLEX I ENZYME ACTIVITY OF MELAS CYBRID CELLS

### Materials and Methods

Complex I enzyme activity was measured at 37° C. on an UVmc2 spectrophotometer (SAFAS) as described (Desquiret-Dumas et al., 2012). For complex I enzyme activity, 0.5 million of cells were sonicated (6 cycles of 5 seconds) then incubated at 37° C. in the reaction medium (KH₂PO₄ 100 mM, pH 7.4, KCN 1 mM, NaN₃ 2 mM, BSA 1 mg/ml, ubiquinone-1 0.1 mM and DCPIP 0.075 mM). The reaction was started by adding 0.15 mM NADH and the disappearing rate of DCPIP was measured at 600 nm for 2 minutes. The unspecific activity was determined in the presence of rotenone (5 μM).

### Results

FIG. 5 shows that DSF at various concentrations, especially from 30 to 90 nM, increases complex I activity in MELAS neuronal mutant cybrid cells.

### EXAMPLE 7: EFFECT OF DSF ON MITOCHONDRIAL COMPLEX I RESPIRATION ON MELAS CYBRID CELLS

### Materials and Methods

Cellular oxygen consumption was measured at 37° C. in MELAS cybrid cells treated vs untreated mutant cells on a high-resolution oxygraph (Oroboros), as described elsewhere (Desquiret-Dumas et al., 2012).

Briefly treated and untreated mutant cells were trypsinized and the pellet was resuspended in the respiratory buffer (KH₂PO₄ 10 mM, mannitol 300 mM, KCl 10 mM, MgCl₂ 5 mM, pH 7.4) containing 15 μg/million cells of digitonin. Cells were incubated at room temperature during 2.5 minutes and the digitonin action was stopped by adding five volumes of the respiration buffer supplemented with 1 mg/ml of BSA. Cells were centrifuged (800 rpm, 2.5 minutes), resuspended in the respiration buffer+BSA (50 μl/million of cells) and placed in the oxygraph chamber (Oroboros). The oxygen consumption was measured in state II (5 mM malate+pyruvate), state III (5 mM malate+pyruvate+1.5 mM ADP+0.5 mM NAD or 5 mM succinate+10 μM rotenone+1.5 mM ADP+0.5 mM NAD), state IV (8 μg/ml oligomycin) and maximal cytochrome c oxidase capacity (4 mM ascorbate+0.2 mM TMPD). After oxygraphic measurement, 400 W of cell suspension was removed from the chamber and the protein concentration was measured using bicinchoninic acid.

### Results

FIG. 6 shows that DSF at various concentrations increases complex I linked respiration in MELAS neuronal cybrid cells.

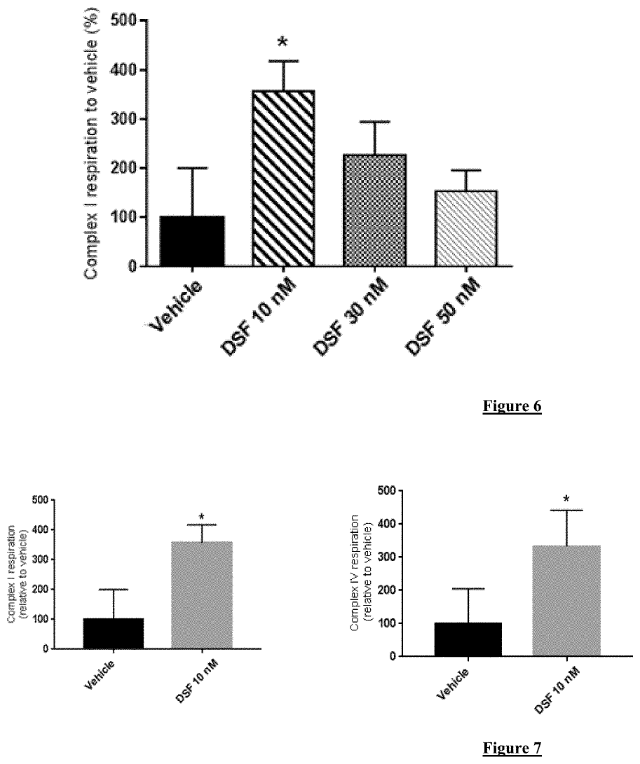

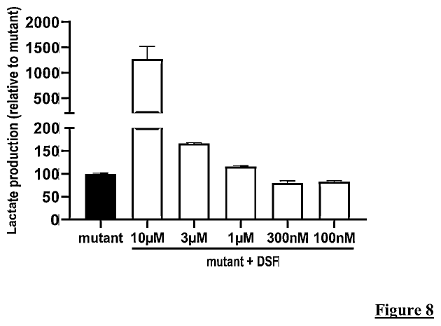

Moreover, FIG. 7 reveals that DSF at concentration 10 nM increases mitochondrial complex I as well as complex IV linked respiration in MELAS neuronal cybrid cells.

### EXAMPLE 8: DETERMINATION OF THE MAXIMAL DSF CONCENTRATION NONTOXIC FOR LACTATE PRODUCTION IN MELAS CYBRID CELLS

### Materials and Methods

### Lactate Measurements

Lactate concentrations in the culture media were determined by spectrophotometry on a Hitachi-Roche apparatus following the recommendations of the manufacturer (Roche Diagnosis, Bale, Switzerland). Increased lactate concentration in supernatant of cell culture is witnessing glycolytic adaptation at the expense of mitochondrial function with reduced oxidative mitochondrial metabolism correlated with high concentrations and drug toxicity of DSF.

### Results

Results are shown in FIG. 8.

FIG. 8 reveals that DSF, at concentrations lower than 1 μM, has no impact on lactate production of MELAS cybrids to evaluate drug toxicity, contrary to the 1 μM or even to higher concentrations 3 μM or 10 μM.

### EXAMPLE 9: EFFICACY OF A DSF TREATMENT IN A MURINE MODEL

A study is performed using the murine model ND6mut which harbors the homoplasmic m.13997G<A of mitochondrial DNA (mtDNA) for the ND6 gene. Said gene encodes the NADH-ubiquinone oxidoreductase chain 6 protein which is a subunit of the respiratory chain Complex I. The corresponding human mutation is m.14600G<A, leading to the substitution pPro25Leu. Such a genetic variant was reported in humans as being responsible for mitochondrial diseases. The ND6mut mice display encephalopathic disorders, optical atrophy as well as cardiomyopathy usually starting at the age of 6 months.

The treatment with DSF is applied on 5-month-old mice during 1 month. DSF is administered per os at the following daily doses:
100 mg/kg
50 mg/kg
25 mg/kg
5 mg/kg.

The following tests are performed before and after treatment, on control mice (not mutated for the ND6 gene) as well as on ND6mut mice treated or not with DSF:
assessment of cardiac function by echocardiographic analyses;
assessment of muscular function by treadmill testing and voluntary running-wheel activity (ACTIVIWHEEL);
molecular and histological analysis of isolated hearts (e.g. integrity of the mitochondrial genome and complex I activity).

### REFERENCES

Aiyar R S et al. Nature Communications 2014, 18; 5:5585, PMID: 25519239
Bach S et al., Nature Biotechnology 2003, 21(9):1075-81, PMID: 12910243
Baruffini, E. et al. (2006) Hum. Mol. Genet., 15(19), 2846-55
Couplan E et al. (2011) Proc Natl Acad Sci USA 108(29): 11989-94; PMID: 21715656
De Luca C. et al. (2009) Mitochondrion 9(6): 408-17
Desquiret-Dumas V et al. (2012) Biochimica et Biophysica Acta. 1822(6):1019-29, PMID: 22306605
De Taffin de Tilques et al. (2017) Dis Model Mech. 10(4):439-450, PMID: 28188263
Geffroy G et al., Biochim Biophys Acta. 2018; 1864(5Pt A):1596-1608, PMID: 29454073
Lin et al., Proc Natl Acad Sci USA. 2012, 109(49):20065-70
McManus et al., Cell Metab. 2019, 29(1):78-90
Manfredi G et al. JBC 1999, 274(14): 9386-91, PMID: 10092618

1. A pharmaceutical composition comprising disulfiram or one of its derivatives for use in the treatment of a mitochondrial disease or mitochondrial dysfunction.

2. A composition for its use according to claim 1, wherein the composition comprises disulfiram or sodium diethyldithiocarbamate.

3. A composition for its use according to claim 1 or 2, wherein the mitochondrial disease is a mitochondrial respiratory chain disease.

4. A composition for its use according to any of the preceding claims, wherein the mitochondrial disease is a genetic disease.

5. A composition for its use according to any of the preceding claims, wherein the mitochondrial disease is linked or due to at least one gene defect in at least one of the following genes: MTTL1, ATP6, TAZ, SURF1, POLG, MPV17, OPA1, COA6, ND6 and BCS1L.

6. A composition for its use according to any of the preceding claims, wherein the mitochondrial disease is selected in the group consisting of:
MELAS syndrome, maternally inherited myopathy and cardiomyopathy, NARP syndrome, Leigh syndrome, Barth syndrome, Mitochondrial DNA Depletion Syndrome 4A, Mitochondrial DNA Depletion Syndrome 4B, Mitochondrial recessive ataxia syndrome, Sensory Ataxic Neuropathy Dysarthria and Ophthalmoplegia, Spinocerebellar Ataxia with Epilepsy, Progressive External Ophthalmoplegia, Mitochondrial DNA depletion syndrome-6, Navajo neuropathy, Behr Syndrome, Mitochondrial DNA Depletion Syndrome 14, infantile cardioencephalomyopathy due to cytochrome c oxidase deficiency, Mitochondrial Complex III Deficiency Nuclear Type 1, GRACILE Syndrome and Bjornstad Syndrome.

7. A composition for its use according to any of the preceding claims, wherein the composition is administered orally.

8. A composition for its use according to any of the preceding claims, wherein the composition is administered daily.

9. A composition for its use according to any of the preceding claims, wherein the composition is orally administered at a dosage, advantageously at a daily dosage inferior or equal to 8 mg/kg or inferior or equal to 7, 6, 5, 4, 3, 2, 1 mg/kg, or even inferior or equal to 0.9, 0.8, 0.7, 0.6, 0.5, 0.4, 0.3, 0.2, or 0.1 mg/kg.

10. A composition for its use according to any of the preceding claims, wherein the composition is in a solid form, advantageously in the form of a tablet.

11. A composition for its use according to claim 10, wherein the composition comprises 500 mg of the active compound, in particular disulfiram, advantageously less than 400 mg, 250 mg, 200 mg or even less than 100 mg or 50 mg.

12. A composition for its use according to any of the preceding claims, wherein the composition is associated with other treatments for the same disease.

13. A composition for its use according to any of the preceding claims, wherein the composition comprises another compound for treating the same disease.
